(19) United States
(12) Patent Application Publication
(10) Pub. No.: US 2004/0009233 A1
(43) Pub. Date: Jan. 15, 2004

(54) Shark meat extract

(76) Inventors: Paul Frank Davis, Wellington (NL); Andrew Douglas Mackenzie, Tokyo (JP)

Correspondence Address: NIXON & VANDERHYE, PC, 1100 N GLEBE ROAD, 8TH FLOOR, ARLINGTON, VA, 22201-4714, US

(21) Appl. No.: 10/380,545

(22) PCT Filed: Dec. 18, 2001
(86) PCT No.: PCT/NZ01/00285

(30) Foreign Application Priority Data
Dec. 20, 2000 (NZ) 509047

(51) Int. Cl. A61K 35/56

(52) U.S. Cl. 424/520

(57) ABSTRACT

An extract obtained from shark meat, which extract is capable of inhibiting angiogenesis in an animal. A process for obtaining the extract by contacting shark meat with a solvent, separating the resulting solution from the meat, then removing the solvent to give an extract which is an inhibitor of angiogenesis. The use of the extract for treatment of diseases associated with angiogenesis including cancer, retinopathy, inflammation, and arthritis.

This invention relates to an extract obtained from the meat of a shark and its use for the prevention or inhibition of angiogenesis. In particular, the invention relates to an extract from shark meat which is obtained by solvent extraction followed by removal of the solvent to give an oil which is useful as an angiogenesis inhibitor.

### BACKGROUND

It has been known for some years that various shark products are useful for the prevention or treatment of certain diseases. In particular, powdered shark cartilage has been used in attempts to prevent or control certain cancers.

Angiogenesis (the growth of new blood vessels) is important for the survival and growth of cancer tumours. Such tumours have a high rate of metabolism relative to non-cancerous growths or organs and, consequently, more blood is needed by tumours to meet their higher nutritional requirements. The inhibition of angiogenesis is therefore one mechanism of controlling or preventing the growth of tumours.

Shark cartilage has been shown to inhibit angiogenesis. However, current disadvantages associated with the use of shark cartilage for treating or preventing cancer include the need to ingest substantial amounts of the cartilage. It is considered that the dosage rate needed is at least one gram of cartilage per kilogram of body weight. In addition, shark cartilage has a smell and taste which are undesirable to humans.

Oil obtained from shark liver has also been reported to slow cancer growth. The anti-cancer effect may be due to the presence of alkylglycerols in shark liver oil. Alkylglycerols are also found in milk, blood, and immune system organs such as the liver, spleen, bone marrow, and lymphatic tissue.

Shark liver oil is also known to contain the compound squalamine. Animal trials have reportedly demonstrated that squalamine disrupts the ability of a tumour to develop its own blood supply. It is therefore thought that shark liver oil containing squalamine may be useful in the treatment or prevention of certain cancers.

Chinese patent application no. CN01174036A describes shark meat that has been processed using a method including the steps of dewatering, freezing, and crushing etc. The processed meat has been reported to prevent cancer. There is no description of a process for obtaining an extract from the meat nor is there any description of angiogenesis inhibition.

The above shark products each suffer from the disadvantages that the specific angiogenesis inhibiting activity is low and therefore excessive quantities of product must be ingested, and that shark cartilage and organs, such as shark liver, are relatively expensive raw materials.

Canadian patent application no. 2,201,025 describes a medicine containing substances obtained from a variety of sources, including the flesh of sharks. The medicine is described as being useful for treating diseases such as hepatitis C. There is no indication that the medicine has any angiogenesis inhibiting ability or is effective against any diseases or disorders associated with angiogenesis. Furthermore, the substance derived from shark flesh, which is incorporated into the medicine, is obtained by drying the flesh, heating to release oils from the flesh, and then grinding the flesh. No information is provided as to the composition of the ground flesh or what compounds are removed in the heating step.

The inventors have now found that an extract from shark meat, obtained by solvent extraction, is a surprisingly potent inhibitor of angiogenesis and is therefore a potential agent for the treatment of a number of diseases or disorders related to angiogenesis.

It is therefore an object of this invention to provide an angiogenesis inhibitor obtained by solvent extraction from shark meat, or to at least provide a useful alternative.

### STATEMENTS OF INVENTION

In a first aspect of the invention there is provided an extract obtained from shark meat, which extract is capable of inhibiting angiogenesis in an animal.

The extract of the invention preferably has, as a major component, phospholipids. The phospholipid content is preferred to be 20-40% by weight of the extract. Typical phospholipids are phosphatidylethanolamine (PE) and phosphatidylcholine (PC).

It is further preferred that the phospholipid fatty acids contain a high level of docosahexaenoic acid (DHA), for example 20-35% DHA by weight of the total fatty acid content.

It is also preferred that the extract contains <10% by weight triglycerides in the extract.

In a second aspect of the invention there is provided a process for obtaining an angiogenesis inhibiting extract from shark meat including:

contacting meat from one or more sharks with a solvent for a time sufficient to allow extraction of one or more substances from the meat into solution,

separating the meat from the solution, and

removing the solvent from the solution to give an extract which is an inhibitor of angiogenesis.

Any suitable organic solvent or supercritical CO₂ may be used. Preferably, the solvent is ethanol, methanol, ethyl acetate, or dichloromethane, or a mixture of any such solvents.

In a preferred embodiment of the invention the shark meat is freeze-dried prior to contact with the solvent. Alternatively, the shark meat may be air-dried.

Any means of separating the solvent from the meat may be used. However, separation is preferably by filtration.

The meat from any shark may be used. However, it is preferred that the shark meat is from any one or more of the following: rig (lemonfish), school shark, ghost shark, mako, blue shark, elephant fish, salmon shark, and blacktip reef shark.

In a third aspect of the invention there is provided a pharmaceutical composition containing an angiogenesis inhibiting extract obtained from the meat of at least one shark, together with a suitable carrier.

In a fourth aspect of the invention there is provided the use of an angiogenesis inhibiting extract obtained from shark meat in the manufacture of a medicament for the treatment or prevention of certain diseases or disorders. The diseases or disorders include cancer, retinopathy, inflammation, and arthritis.

In a fifth aspect of the invention there is provided a method of using an angiogenesis inhibiting extract from shark meat for the treatment or prevention of certain diseases or disorders in an animal. The diseases or disorders include cancer, retinopathy, inflammation, and arthritis. Although the invention relates to a variety of animals, it is preferred that the animal is a human.

### DETAILED DESCRIPTION

The term “shark meat” as used herein means any meat or flesh of a shark but, for the avoidance of doubt, is not intended to include any of the internal organs of the shark.

The extract of the invention is typically an oil or oil-like substance, such as a paste, at ambient temperatures, typically in the range approximately 15-25° C. Any reference in this specification to “oil” is intended to include reference to substances which have oil-like or paste characteristics.

A major component of the extract has been found to be phospholipids, such as phosphatidylethanolamine (PE) and phosphatidylcholine (PC). The phospholipid content is typically in the range 20-40% by weight.

The phospholipid fatty acids usually include docosahexaenoic acid (DHA), arachidonic acid (AA), eicosapentaenoic acid (EPA), and others. In contrast to other fish extracts or products, the extract of this invention contains a high level (20-35% by weight) of DHA in the total fatty acid component. As can be seen from the table below, a sample of DHA has been found to be a strong inhibitor of angiogenesis. Given the high level of DHA in the extract of this invention, it is speculated that DHA is the component of the extract that is partly responsible for the angiogenesis inhibiting properties of the extract.

| | | | Sample | | Inhibition of |
|---|---|---|---|---|---|
| Common Name | Structure | | Concentration | | Angiogenesis |
| Monounsaturated | | | | | |
| Myristoleic | 14:1 | (n-5) | 10 | μg/ml | 58% |
| Palmitoleic | 16:1 | (n-7) | 5 | μg/ml | 70% |
| Heptadecenoic | 17:1 | (n-7) | 20 | μg/ml | 83% |
| Oleic | 18:1 | (n-9) | 100 | μg/ml | 0% |
| Petroselenic | 18:1 | (n-12) | 100 | μg/ml | 58% |
| Erucic | 22:1 | (n-9) | 200 | μg/ml | 30% |
| Nervonic | 24:1 | (n-9) | 100 | μg/ml | 0% |
| Polyunsaturated | | | | | |
| Linoleic | 18:2 | (n-6) | 5 | μg/ml | 82% |
| α-Linolenic | 18:3 | (n-3) | 20 | μg/ml | 66% |
| Arachidonic | 20:4 | (n-6) | 5 | μg/ml | 84% |
| Eicosapentaenoic | 20:5 | (n-3) | 3 | μg/ml | 100% |
| (EPA) | | | | | |
| Docosapentaenoic | 22:5 | (n-3) | 4 | μg/ml | 71% |
| Docosahexaenoic | 22:6 | (n-3) | 5 | μg/ml | 88% |
| (DHA) | | | | | |

Strong Inhibitors (>80% at 5 μg/ml): 20:5, 22:6, 20:4, 18:2
Good Inhibitors (>90% at 10 μg/ml): 22:5, 16:1
Moderate Inhibitors (<90% at 20 μg/ml): 18:3, 17:1, 14:1
Poor Inhibitors (<60% at 100 μg/ml): 18:1, 22:1, 24:1

The extract of the invention is also distinct from other fish extracts and products because it has a surprisingly low level (<10% by weight) of triglycerides.

In a typical extraction, pieces of meat from one or more sharks are reduced in size using a blender, freeze dried, and then mixed with a solvent, such as ethanol. The mixture is typically stirred at room temperature for between 1 and 24 hours. The solvent is then removed from the meat by filtration. The step of mixing the meat with solvent is optionally repeated. The solvent is then removed by evaporation to give the shark meat extract as an oil. The oil typically contains a variety of free fatty acids and glycerol-bound fatty acids.

While Example 1 below describes the use of dichloromethane and methanol, it is anticipated that ethanol will be the preferred solvent of choice.

While Example 2 below describes the fatty acid composition of shark meat oil, it is to be appreciated that components other than fatty acids may be responsible for the angiogenesis inhibiting activity of the shark meat oil.

Example 3 describes the extraction of oil from freeze dried blue shark flesh. Freeze-dried shark powder was extracted with EtOH and with EtOAc. A significant difference between the yields of oil from the two extractions (13.7% for EtOH, 1.6% for EtOAc) resulted. Although the yield of the EtOAc extract is lower, the specific angiogenesis inhibiting activity of this extract is higher indicating a higher concentration of inhibiting compounds in that extract.

As can be seen from the bioassay results of Example 3, shark meat oil is a significantly more potent angiogenesis inhibitor than shark cartilage, a solvent extract of shark cartilage, and other fish oils.

The oils from the 3 extractions were analysed for fatty acid composition (by gas chromatography), and phospholipid composition (by ³¹P NMR). Thin layer chromatography (TLC) was also used to qualitatively analyse the various lipid classes present in the oils. The shark flesh oils were low in non-polar lipids (no triglyceride detected on TLC), with high levels of phospholipids (24% EtOH, 34% EtOAc). The low levels of fatty acid in the shark oils (free or glycerol-bound) suggested a high level of unidentified material in these extracts.

The extract of the invention may be used in the form it is recovered from the process of the invention. However, the extract is preferably mixed with an ingestible oil, such as olive oil, and then encapsulated for oral administration. Alternatively, the extract may be mixed with a solid carrier, such as cyclodextrin, and formed into a tablet, granules, or powder.

Granules or powder or other similar forms may be encapsulated or mixed with another substance, such as food, for ease of oral administration. It is also to be appreciated that the extract can be dissolved in a solvent suitable for administration by injection. Furthermore, an additional ingredient, such as vitamin E, may be included in the formulation.

Angiogenesis is implicated in a wide range of diseases or disorders. The extract of the invention may therefore be useful for the treatment or prevention of any such diseases or disorders. These include, but are not limited to, cancer, retinopathy, inflammation, and arthritis.

Further, while the extract is anticipated to be most useful in the treatment or prevention of diseases or disorders in humans, it is to be appreciated that other animals may benefit from administration of the extract.

### EXAMPLES

The invention is further described with reference to the following examples. It will be appreciated that the invention is not to be construed as limited to the examples.

### Example 1

Extraction of Lemonfish

Lemonfish meat (1.5 kg) was reduced to small pieces using a blender and stirred overnight in a 5 L conical flask with dichloromethane (1.5 L) and methanol (3 L). The solvent was removed from the meat by filtration.

Further dichloromethane (1.5 L) was added to the meat and stirred overnight. The solvent was again removed by filtration and combined with the filtrate of the initial extraction. A salt solution (0.88% KCl, 1.5 L) was added to the solvent and the mixture shaken. The mixture was then allowed to stand and, following separation into two phases, the lower (dichloromethane) phase was recovered and the solvent removed by rotary evaporation to give an oil (12 g, 0.8% yield).

### Example 2

Fatty Acid Composition of Example 1 Oil

The fatty acid composition of the oil prepared according to Example 1 was analysed by gas chromatography (GC).

Before analysis, the fatty acids (free fatty acids and triglyceride fatty acids) were converted to fatty acid methyl esters (FAMEs). The oil (20 mg) was dissolved in hexane (0.5 ml) and added to 1% H₂SO₄ methanol in a seated test tube. The test tube was placed in a water bath at 50° C. overnight. Hexane (2 ml) and 5% aqueous sodium chloride solution (2 ml) were then added and the organic phase (containing the FAMEs) was removed. The organic phase was then washed with 2% sodium bicarbonate solution (2 ml).

GC analysis was carried out using a Hewlett-Packard 5890 GC equipped with an EC-Wax (Alltech) column (30 m×0.25 mm×0.25 μm) with an inlet pressure of 10 psi. The oven temperature was held at 165° C. for 3 min then heated at 4° C./min to 195° C. and held for 10 min. The temperature was then raised at 4° C./min to 225° C. with a final holding time of 12 min. FAMEs were detected using a flame ionisation detector (FID). Peaks were identified by comparison of retention times with those of fatty acid standards and fatty acids contained in previously well characterised natural oils (e.g. cod liver oil).

The results of this analysis are shown in Table 1. Shark meat oil has high levels of docosahexaenoic acid (DHA).

TABLE 1
Fatty acid composition of shark meat oil

| Fatty Acid | % |
|---|---|
| 14:0 | 0.2 |
| 16:0DMA* | 3.4 |
| 16:0 | 19.9 |
| 16:1 | 0.7 |
| 17:0 | 0.5 |
| 17:1 | 0.1 |
| 18:0DMA* | 0.5 |
| 18:1DMA* | 1.1 |
| 18:0 | 9.6 |
| 18:1 | 8.2 |
| 18:2 | 1.0 |
| 20:1 | 0.5 |
| 20:4 | 6.0 |
| 20:5 | 2.1 |
| 22:4 n-6 | 2.9 |
| 22:5 n-6 | 1.0 |
| 22:5 n-3 | 5.2 |
| 22:6 n-3 | 33.3 |

*dimethylacetal, formed from the vinyl ethers of plasmalogens (phospholipids)

### Example 3

Extraction of Blue Shark

Frozen blue shark fillets were cut into ^{˜}2 cm thick slices and placed in a vacuum oven at 35° C. and at <2 mbar. After 24 hr, the slices were removed and broken into smaller pieces and returned to the vacuum oven for 3-4 days. The shark flesh typically lost 80% of its weight through freeze-drying. The freeze-dried pieces were further broken up by hand and fed into a Waring blender, producing a mixture of dry powder and fibres.

Two extraction experiments were conducted, one using ethanol as the solvent and the other using ethyl acetate.

Shark powder (900 g) and solvent (EtOH or EtOAc, 4.5 L) was stirred overnight. A solvent:powder ratio of 5:1 was needed for efficient stirring. The container was protected from light by wrapping in aluminium foil. The mixture was filtered (through Schleicher & Schuell 595 filter paper). The filtered residue was stirred with fresh solvent (3 L) overnight and the mixture again filtered. The filtrates from the two extractions were combined and the solvent removed by rotary evaporation.

### Example 4

Fatty Acid Composition of Example 3 Oils

The fatty acids (free and bound) in each extract were converted to methyl esters and analysed using GC as described for Example 2.

The EtOH extraction of dried blue shark powder resulted in a high yield of oil (13.7%). In contrast, the EtOAc extraction resulted in only a 1.6% yield.

TABLE 2
Extraction of blue shark powder

| | EtOH | EtOAc |
|---|---|---|
| Yield from | 13.7% | 1.6% |
| powder | | |
| Yield from | 2.7% | 0.3% |
| wet flesh | | |
| Appearance | Off-white | Orange/ |
| | opaque oil | brown |
| | | semi-solid |

Thin layer chromatography was used to show the different lipid classes present in the extracts. Each extract showed very little on the non-polar TLC plate (cholesterol and faint FFA spots, no sign of triglycerides), but high levels of polar lipids, for example phosphatidylethanolamine (PE), phosphatidylcholine (PC), and sphingomyelin (SM).

The shark extracts exhibited significant differences in fatty acid content and composition (Table 3). The total fatty acid content of the EtOAc extract was 30.1%. In comparison, the EtOH extract contained a significantly lower total level of fatty acids at 7.3%. It is clear from this result, together with the greater total yield of the EtOH extraction, that EtOH extracts more non-lipid material than EtOAc.

The fatty acid compositions of the extracts were quite similar with both having high levels of docosahexaenoic acid (DHA) 20.0-22.2%, oleic acid (18:1) 10.0-12.2%, stearic acid (18:0) 11.2-14.9%, and palmitic acid 16.1-19.1%.

TABLE 3
Fatty acid composition (wt/wt %) of blue shark extracts.

| | | Retention | | |
|---|---|---|---|---|
| | Fatty Acid | time (min) | EtOH | EtOAc |
| | 16:0DMA* | 9.40 | 10.1 | 4.5 |
| | 16:0 | 10.07 | 19.1 | 16.1 |
| | 16:1 | 10.59 | 2.0 | 1.7 |
| | 18:0DMA* | 13.74 | 1.4 | 0.9 |
| | 18:1DMA* | 14.24 | 4.6 | 2.3 |
| | 18:0 | 14.85 | 11.2 | 14.9 |
| | 18:1 (n-9) | 15.53 | 12.2 | 10.0 |
| | 18:1 (n-7) | 15.74 | 4.9 | 4.5 |
| | 18:2 (n-6) | 17.16 | | 1.2 |
| | 20:1 (n-11) | 23.45 | | 1.3 |
| | 20:1 (n-9) | 23.60 | | 1.7 |
| AA | 20:4 (n-6) | 27.04 | 3.6 | 6.0 |
| EPA | 20:5 (n-3) | 29.15 | 5.9 | 6.7 |
| | 22:5 (n-3) | 37.53 | 5.0 | 4.8 |
| DHA | 22:6 (n-3) | 39.10 | 20.0 | 22.3 |

| | | |
|---|---|---|
| Total fatty acid content (%) | 7.3 | 30.1 |

*dimethylacetyl, formed from the vinyl ethers of plasmalogens (phospholipids)
AA = arachidonic acid
EPA = eicosapentaenoic acid
DHA = docosahexaenoic acid

### Example 5

Phospholipid Composition of Example 3 Oils

Phospholipids were characterized using ³¹P NMR. Hexamethylphosphoramide (HMPA) was used as an internal standard for quantification. ³¹P NMR confirmed the observation by TLC that the oils contain high levels of phospholipids. The oils had significant total levels of phospholipids (24% for the EtOH extraction and 34% for the EtOAc extraction).

TABLE 4
Phospholipid composition (wt/wt %) of the blue shark oils

| | | | | Shark EtOH |
|---|---|---|---|---|
| | Shift (ppm) | Shark | Shark | (large |
| Phospholipid | (relative to PC) | EtOH | EtOAc | scale) |
| PG | 1.24-1.27 | 2.2 | 1.4 | 1.8 |
| PI | 1.52-1.60 | 1.7 | 5.6 | 1.9 |
| SM | 0.79-0.83 | 6.9 | 9.7 | 7.5 |
| PE | 0.56-0.63 | 22.0 | 31.8² | 20.8 |
| MPE/LPC? | 0.46-0.48 | 5.7 | | 3.2 |
| DPE? | 0.19-0.28 | 5.0 | 6.2 | 4.3 |
| AAPC | 0.06 | | 8.2 | 9.1 |
| PC | 0 | 56.5¹ | 37.17 | 51.5 |

¹PC + AAPC
²PE + MPE/LPC?
PI = phosphatidylinositol
SM = sphingomyelin
PE = phosphatidylethanolamine
MPE = monomethylphosphatidylethanolamine
LPC = lysophosphatidylcholine
DPE = dimethylphosphatidylethanolamine
AAPC = alkylacylphosphatidylcholine
PC = phosphatidylcholine

### Example 6

In vitro Bioassays

The oil prepared according to Example 1 was assessed for antiangiogenic activity using an aortic ring assay. The method is based on that described by Nicosia and Ottinetti (Lab Invest. 63: 115-122 (1990)) and by Brown et al (Lab Invest. 75: 539-555 (1996)). Following removal of fat and perivascular fibrous tissue, rat aorta was cut into rings about 2 mm in thickness. A plug of fibrin gel (0.4 ml) (prepared by adding thrombin to fibrinogen solution dissolved in MCDB131 medium) was formed in wells of a 24-well culture plate. An aortic ring was placed in the centre of each well and overlaid with another plug of fibrin (0.4 ml). Each gel was covered with MCDB131 medium (1.5 ml) and incubated at 37° C. in an atomsphere of 3%CO₂/97% air. Shark meat extracts to be tested were added as supplements to the medium. Each such extract was assayed in triplicate.

After approximately five days, microvessels could be detected growing from the perimeter of the rings. At regular intervals between five and fourteen days, images of each well were recorded using a digital camera attached to an inverted microscope. The area of microvessel growth relative to the perimeter of the ring for each image was determined using NIH Image 1.59 software. At each time point a mean value for the growth rate was determined and the rate of microvessel growth was then calculated for each shark meat extract.

Compared with commercially available fish oils and other shark products, shark meat oil displayed higher angiogenic activity as shown in Table 5.

TABLE 5
Antiangiogenesis activity of shark products and fish oils

| | Angiogenesis Inhibition | | | |
|---|---|---|---|---|
| Sample | 1 mg/ml | 0.1 mg/ml | 0.04 mg/ml | 0.01 mg/ml |
| Shark Meat Oil | | 100% | 90% | 73% |
| Shark Cartilage | 73% | | | |
| Shark Cartilage | | 57% | | |
| (solvent extract) | | | | |
| Fish Oil | | 88% | | |
| Cod Liver Oil | | 42% | | |
| Shark Liver Oil | | 24% | | |

Aliquots of each of the Example 3 oils (as well as an oil obtained by extraction with methanol) were assayed for their ability to modulate angiogenesis using the rat aortic ring model similar to that used above for the Example 1 oil.

Each sample was assayed in triplicate and the results are the mean of these replicates. A control set of three wells was run, which has only the carrier added. Growth rates in the presence of the test materials were assessed relative to this. Inhibition of angiogenesis was assessed at several concentrations of oils obtained with different of organic solvents, as shown in Table 6.

TABLE 6
Inhibition of angiogenesis by extracts from shark flesh

| | Concentration (μg/ml) | % Inhibition |
|---|---|---|
| Ethanol Extract | 10 | 84.9 |
| | 5 | 79.8 |
| | 1 | 38.2 |
| Methanol Extract | 10 | 98.2 |
| | 3 | 71.0 |
| | 1 | 50.8 |
| Ethyl Acetate Extract | 2 | 100 |
| | 1 | 96.4 |
| | 0.25 | 65.1 |

These experiments indicate that the ethanol and methanol extracts give very similar levels of inhibition of angiogenesis with approximately 50% inhibition in the 1 to 3 μg/ml concentration. The ethyl acetate extract is about 5 times more potent with 50% inhibition being at about 0.2 μg/ml.

The ethanol extract was mixed with olive oil and its effect on angiogenesis measured in the aortic ring assay. Firstly, olive oil is not inhibitory even at relatively high concentrations. In fact at 200 μg/ml it is slightly pro-angiogenic eliciting a 33.6% stimulation.

When the ethanol extract was mixed with olive oil at a ratio of 1 part extract to 4 parts olive oil (vol/vol) and assayed at 15 μg/ml (3 μg of extract/ml), an inhibition of 87.5% was measured.

When the ethanol extract was mixed with olive oil at a ratio of 1 part extract to 9 parts olive oil (vol/vol) and assayed at 30 μg/ml (3 μg of extract/ml), an inhibition of 85.8% was measured.

When a paste of ethanol extract was mixed with β-cyclodextrin at a ratio of 1 part of extract to 6 parts of β-cyclodextrin (wt/wt), a 40% inhibition of angiogenesis was measured when incubated at a concentration of 15 μg/ml (2.5 μg of extract/ml).

### Example 7

In Vivo Bioassays

Extracts from shark meat were incorporated into drinking water made available to rats. Each extract was dissolved in the water at 0.166 mg/ml and changed for fresh supplemented water every second day. The consumption was measured at each change and the dosage of each extract determined. Both an ethanol and an ethyl acetate extract of shark meat were evaluated. The control rats had unsupplemented water freely available. Each group comprised six Sprague-Dawley rats (3 male, 3 female).

Two weeks after commencing the administration of the extract supplement, the induction of angiogenesis was initiated. This was via Compound 48/80 delivered in increasing doses twice daily intra-peritoneally over 4.5 days (as described by Davis, et. al., Microvasc. Res., 54: 178-182 (1997)).

Sixteen days after the commencement of the Compound 48/80 injections, the vascular system of each rat was highlighted with India ink and the gut and associated mesenteric windows were excised and spread out on glass slides and dried. Images of these slides were recorded digitally and the proportion of the area of each window occupied by microvessels was calculated.

The mean value for the vascularisation for each group was determined and the statistical significance of this assessed by the Student t-test.

The body weights of the groups of rats were measured at the time points indicated in Table 7.

TABLE 7
Body Weight in grams (% increase)

| | | Commencement | |
|---|---|---|---|
| | Commencement | of | |
| | of | Compound 48/80 | Conclusion of |
| | Supplementation | Administration | of Experiment |
| CONTROL | | | |
| Male | 202 | 290 (43.6%) | 331 (63.9%) |
| Female | 160 | 203 (26.9%) | 240 (50.0%) |
| SHARK FLESH | | | |
| ETHANOL | | | |
| EXTR | | | |
| Male | 223 | 310 (39.0%) | 355 (59.2%) |
| Female | 170 | 219 (28.8%) | 253 (48.8%) |
| SHARK FLESH | | | |
| ETHYL | | | |
| ACETATE | | | |
| EXTR | | | |
| Male | 210 | 298 (41.9%) | 351 (67.1%) |
| Female | 156 | 200 (28.2%) | 234 (50.0%) |

The results in Table 7 show a negligible difference in the rate of increase in body weight for rats supplemented with either of the extracts. These supplemented animals had similar growth rates to that of the Control group. This comment applies to both male and female rats, with the rate of growth of the males being greater. The supplementation with shark meat extracts has no significant effect on the growth rate of the rats.

The consumption of extract relative to body weight was calculated. The calculation was based on the measured body weight of the rats and on the measured water consumption by the rats.

TABLE 8
Daily dose of shark flesh lipid (mg per kg body weight)

| | Mean | Range |
|---|---|---|
| SHARK FLESH | | |
| ETHANOL EXTR | | |
| Male | 7.86 | 4.07-9.88 |
| Female | 8.74 | 4.31-10.04 |
| SHARK FLESH | | |
| ETHYL ACETATE EXTR | | |
| Male | 6.92 | 4.56-8.77 |
| Female | 7.25 | 3.85-9.39 |

The highest dosage was measured at the commencement of the experiment. During the administration of Compound 48/80, the water consumption by the rats dropped significantly indicating reduced extract consumed. The dosage is therefore lowest during this period. Following completion of the Compound 48/80 administration the water consumption, and consequently the extract consumption, increased.

In vivo angiogenesis was measured in the mesenteric windows as described above. The results are presented in Table 9 below.

The ethanol extract caused a significant inhibition of angiogenesis (46%) at this dosage. In comparison, the ethyl acetate extract resulted in only 21% inhibition at approximately the same dosage. This is still significant. However, although the dosages were about the same for both extracts, it is known that the ethyl acetate extract was considerably more inhibitory in the in vitro aortic ring assay.

TABLE 9
Inhibition of Angiogenesis by Shark Flesh Extracts

| | % Inhibition |
|---|---|
| SHARK FLESH | |
| ETHANOL EXTR | |
| All animals | 45.78% (n = 77) |
| Male | 54.31% (n = 38) |
| Female | 27.79% (n = 39) |
| SHARK FLESH | |
| ETHYL ACETATE EXTR | |
| All animals | 20.87% (n = 77) |
| Male | 24.49% (n = 31) |
| Female | 10.50% (n = 46) |

### Example 8

Anti-Inflammation Assay

For assessing the effect of the ethanol extract of shark meat on acute inflammation, an ethanolic extract was administered orally by inclusion in the drinking water for each of six rats (3 male, 3 female). This was for one week prior to the initiation of acute inflammation. The control group did not receive this supplementation. The dosage was at 0.2 mg of extract per ml of drinking water.

Inflammation was induced by injecting 100 μl of a 2.5% γ-carrageenan solution into both hind footpads of each rat. The volume displacement of each foot was measured prior to the injection and then again after 4 hours. The volume change for each foot was determined.

The consumption of extract averaged 4.67 mg per day for each male rat and 3.75 mg per day for each female rat.

For the 6 rats (1 2 feet) which received a normal (unsupplemented) diet, the mean footpad volume increase was 54.21%±2.30(SEM). For the 6 rats (12 feet) that received the extract the mean footpad volume increase was 47.06%±2.27(SEM). This indicates that the extract inhibited the acute inflammatory response by 13.19%±0.20 (SEM). Thus, it appears that the extract does have some anti-inflammatory reactivity.

### Example 9

Formuation Examples

Shark meat extract in olive oil-shark meat extract, obtained by ethanol extraction, and vitamin E oil were added to olive oil. The mixture (105 mg) was encapsulated in a soft gelatin capsule, the mixture comprising extract (10 mg), vitamin E oil (5 mg), and olive oil (90 mg).

Shark meat extract and cyclodextrin-shark meat extract (15 mg) was mixed with beta-cyclodextrin (85 mg) and vitamin E (0.075 mg). The mixture was further processed to give a powder or granules ready for use.

Although the invention has been described by reference to examples, it should be appreciated that variations and modifications may be made without departing from the scope of the invention. Furthermore, where known equivalents exist to specific features, such equivalents are incorporated as if specifically described in this specification.

Industrial Applicability

The extract of the invention is an inhibitor of angiogenesis. Angiogenesis is implicated in a variety of diseases or disorders. The inhibition of angiogenesis may therefore be one means for preventing or treating such diseases or disorders. The diseases or disorders include cancer, retinopathy, inflammation, and arthritis. The extract of the invention is therefore useful for treating at least these diseases.

1. An extract obtained from shark meat, which extract is capable of inhibiting angiogenesis in an animal.

2. An extract as claimed in claim 1 which has a phospholipid content of 20-40% by weight of the total extract.

3. An extract as claimed in claim 1 or claim 2 where the phospholipid fatty acid content contains 20-35% by weight docosahexaenoic acid (DHA).

4. An extract as claimed in any one of the preceding claims which has a triglyceride content of less than 10% by weight of the total extract.

5. An extract as claimed in any one of the preceding claims where the shark meat is obtained from any one or more of the following: lemonfish, school shark, rig, ghost shark, mako, blue shark, elephant fish, salmon shark, and blacktip reef shark.

6. A process for obtaining an angiogenesis inhibiting extract from shark meat including:
contacting meat from one or more sharks with a solvent for a time sufficient to allow extraction of one or more substances from the meat into solution,
separating the meat from the solution, and
removing the solvent from the solution to give an extract which is an inhibitor of angiogenesis.

7. A process as claimed in claim 6 where the solvent is an organic solvent or is supercritical CO₂.

8. A process as claimed in claim 7 where the organic solvent is ethanol, methanol, ethyl acetate, or dichloromethane, or a mixture of any such solvents.

9. A process as claimed in any one of claims 6 to 8 where the shark meat is air-dried or freeze-dried prior to contact with the solvent.

10. A process as claimed in any one of claims 6 to 9 where the meat is separated from the solution by filtration.

11. A process as claimed in any one of claims 6 to 10 where the shark meat is obtained from any one or more of the following: rig (lemonfish), school shark, ghost shark, mako, blue shark, elephant fish, salmon shark, and blacktip reef shark.

12. A pharmaceutical composition containing an angiogenesis inhibiting extract obtained from the meat of at least one shark, together with a suitable carrier.

13. A composition as claimed in claim 12 which is a solution or suspension of the extract in an edible oil.

14. A composition as claimed in claim 13 where the oil is olive oil.

15. A composition as claimed in claim 14 which is a mixture of the extract and cyclodextrin.

16. The use of an angiogenesis inhibiting extract obtained from shark meat in the manufacture of a medicament for the treatment or prevention of diseases or disorders.

17. The use as claimed in claim 16 where the diseases or disorders include cancer, retinopathy, inflammation, and arthritis.

18. A method of using an angiogenesis inhibiting extract obtained from shark meat for the treatment or prevention of diseases or disorders in an animal.

19. A method as claimed in claim 18 where the diseases include cancer, retinopathy, inflammation, and arthritis.

20. An extract as claimed in claim 1 substantially as herein described with reference to any example thereof.

21. A process as claimed in claim 6 substantially as herein described with reference to any example thereof.

22. A pharmaceutical composition containing an extract as claimed in claim 1 substantially as herein described with reference to the Formulation Examples.

23. The use as claimed in claim 16 substantially as herein described.

24. A method as claimed in claim 18 substantially as herein described.
